SEQUENCE LISTING (10 sequences)

SEQ ID NO: 1 - DNA, 22 bp, Artificial Sequence
  The sequence is synthetized.
atggattcttctggtgtcctct

SEQ ID NO: 2 - DNA, 21 bp, Artificial Sequence
  The sequence is synthetized.
ttggttgctgctcaaacctag

SEQ ID NO: 3 - DNA, 1038 bp, Atropa belladonna
atggattcttctggtgtcctctctccttttacattttcaagaagggcaactggtgaagaagatgtgaggttgaaggtactttattgtgggatttgtcactcagatctgggttgcatcaagaatgaatggggctggtgttcataccctcttgtacctggccatgagattgttggcatagcaacagaggtgggaagcaaagtgacaaaattcaaagttggagacagagtaggagtaggatgcatggttggatcctgtggcacttgccaaaactgcacacaaaatcaagaaagttactgtcctgaagttataatgacttgtgcttctgcttaccctgatggaacaccaacatatggaggattttcaaatcaaatggtagcaaatgagaagtttgtaattcgaatcccaaattcacttccacttgatgctgctgcaccattgctctgtgctggaagtactgtgtatagtgctatgaagttttatggattgtgtagtcaagggttgcacttgggagttgttggtttaggtggacttggtcatgttgctgtgaaatttgcaaaggcatttgggatgaaagtgactgtgattagtacttcacttggaaaaaaagaagaagcaattaatcaacttggagctgattcattcttgatcaatactgatacagagcaaatgcagggtgccatggaggtaatggatggcattattgatacagtatctgcacttcatccaattgaaccattgcttggtctactcaaatctcatcaagggaaactcatcattgttggattacccaataagcaacctgagctgccagtcttttccttgattaatgggaggaaaatgattggagggagtgctgttggaggagtgaaagagacacaagaaatgatagattttgcagcagagcataatatcactgcagacattgagattgtccccatggattatgtgaacacagccatggaacgtcttgagaagggcgatgtcaaatttcgatttgtcattgatgttgaaaatacattggttgctgctcaaacctag

SEQ ID NO: 4 - protein, 345 aa, Atropa belladonna
MDSSGVLSPFTFSRRATGEEDVRLKVLYCGICHSDLGCIKNEWGWCSYPLVPGHEIVGIATEVGSKVTKFKVGDRVGVGCMVGSCGTCQNCTQNQESYCPEVIMTCASAYPDGTPTYGGFSNQMVANEKFVIRIPNSLPLDAAAPLLCAGSTVYSAMKFYGLCSQGLHLGVVGLGGLGHVAVKFAKAFGMKVTVISTSLGKKEEAINQLGADSFLINTDTEQMQGAMEVMDGIIDTVSALHPIEPLLGLLKSHQGKLIIVGLPNKQPELPVFSLINGRKMIGGSAVGGVKETQEMIDFAAEHNITADIEIVPMDYVNTAMERLEKGDVKFRFVIDVENTLVAAQT

SEQ ID NO: 5 - DNA, 31 bp, Artificial Sequence
  The sequence is synthetized.
cgcgaattcatggattcttctggtgtcctct

SEQ ID NO: 6 - DNA, 30 bp, Artificial Sequence
  The sequence is synthetized.
cgcctcgagctaggtttgagcagcaaccaa

SEQ ID NO: 7 - DNA, 29 bp, Artificial Sequence
  The sequence is synthetized.
cgcaagcttctgagttcggatctaggtca

SEQ ID NO: 8 - DNA, 30 bp, Artificial Sequence
  The sequence is synthetized.
cgcggatccttcttcacttttggccttgct

SEQ ID NO: 9 - DNA, 31 bp, Artificial Sequence
  The sequence is synthetized.
cgcggatccatggattcttctggtgtcctct

SEQ ID NO: 10 - DNA, 30 bp, Artificial Sequence
  The sequence is synthetized.
cgcgagctcctaggtttgagcagcaaccaa

(19) United States
(12) Patent Application Publication
(10) Pub. No.: US 2023/0039694 A1
(43) Pub. Date: Feb. 9, 2023

(54) HYOSCYAMINE ALDEHYDE REDUCTASE AND USES THEREOF

(71) Applicant: Southwest University, Chongqing (CN)

(72) Inventors: Zhihua LIAO, Chongqing (CN); Min CHEN, Chongqing (CN); Chunxian YANG, Chongqing (CN); Fei QIU, Chongqing (CN); Junlan ZENG, Chongqing (CN)

(21) Appl. No.: 17/788,105

(22) PCT Filed: Sep. 3, 2020
(86) PCT No.: PCT/CN2020/113138

§ 371 Date: Jun. 22, 2022

(30) Foreign Application Priority Data
Sep. 3, 2019 (CN) 201910826205.8

(51) Int. Cl. C12N 15/82; C12N 9/02

(52) U.S. Cl. CPC C12N 15/8243; C12N 9/0008; C12Y 101/01021

(57) ABSTRACT

Disclosed in the present invention is a hyoscyamine aldehyde reductase (HAR) and uses thereof. The hyoscyamine aldehyde reductase has an amino acid residue sequence as shown in SEQ ID NO. 4 and a nucleotide sequence as shown in SEQ ID NO. 3. After prokaryotic expression of the hyoscyamine aldehyde reductase, a product of a catalyzed hyoscyamine aldehyde reduction reaction is hyoscyamine. After the hyoscyamine aldehyde reductase is used for converting Atropa belladonna, the content of hyoscyamine in an Atropa belladonna cell line can be increased, which has important significance in increasing the content of tropane alkaloids in Atropa belladonna.

The instant application contains a Sequence Listing which has been submitted electronically in the ASCII text file and is hereby incorporated by reference in its entirety. The ASCII text file is a sequence listing entitled “2022-09-27-SequenceListing” created on Sep. 27, 2022 and having a size of 5,747 bytes in compliance of 37 CFR 1.821.

### TECHNICAL FIELD

The present invention relates to the technical field of biology, particularly relates to a hyoscyamine aldehyde reductase, and further relates to uses of the hyoscyamine aldehyde reductase.

### BACKGROUND

Tropane alkaloids (TAs) are a class of anticholinergic drugs with great medical value, which are widely used for anesthetization, pain alleviation, cough and asthma relieving, and motion sickness resistance, and further used for controlling rigidity and tremor of the Parkinson's disease. Clinically common tropane alkaloids are hyoscyamine and scopolamine, the market demands of which are very great, and scopolamine is weaker in toxic and side effect, stronger in pharmaceutical effect and further higher in cost. At present, TAs are all extracted from Solanaceae resource plants containing TAs, including Atropa belladonna, Datura stramonium and Hyoscyamus niger. Atropa belladonna is a main commercially cultivated medicine resource of scopolamine and hyoscyamine and also a medicinal plant of TAs included in Pharmacopeia. In a wild-type Atropa belladonna plant, the content of hyoscyamine is 0.02%-0.17% (by dry weight), and the content of scopolamine is very low, being only 0.01%-0.08% of the total dry weight. Therefore, breeding Atropa belladonna with a high yield of tropane alkaloids has long been a goal of the industry.

The content of most plant secondary metabolites in natural plants is extremely low, while a chemical synthesis method used is complex in process flow and excessively high in cost; and in addition, biosynthesis pathways of many plant secondary metabolites are not clear, so total chemical synthesis cannot be achieved. Therefore, researchers began to explore other methods for increasing the content of plant secondary metabolites. For example, key enzyme genes in biosynthesis of secondary metabolites are over-expressed in a plant, and a rate-limiting step of synthesis of the metabolites is broken down, thereby facilitating accumulation of final useful metabolites in the plant and obtaining materials with higher economic value. In Atropa belladonna, after over-expression of an H6H gene, a large quantity of hyoscyamine in Atropa belladonna is converted to more valuable scopolamine, which greatly increases the economic value of Atropa belladonna. Hyoscyamine is an important anticholinergic drug and is also an immediate precursor of scopolamine. The use of plant secondary metabolic engineering relies on analysis on biosynthesis pathways of secondary metabolites. Therefore, cloning a biosynthesis gene of hyoscyamine has important significance in improving and increasing the content of tropane alkaloids in Atropa belladonna.

### SUMMARY

In view of this, a first objective of the present invention is to provide a hyoscyamine aldehyde reductase; a second objective of the present invention is to provide a hyoscyamine aldehyde reductase gene; a third objective of the present invention is to provide a recombinant expression vector containing the hyoscyamine aldehyde reductase gene; a fourth objective of the present invention is to provide a transgenic cell line or transgenic recombinant bacteria containing the hyoscyamine aldehyde reductase gene; a fifth objective of the present invention is to provide a use of the hyoscyamine aldehyde reductase in catalyzing reduction of hyoscyamine aldehyde to produce hyoscyamine in vitro or in vivo; a sixth objective of the present invention is to provide uses of the hyoscyamine aldehyde reductase gene and the recombinant expression vector in reconstructing a synthesis pathway of hyoscyamine in a prokaryote or a eukaryote not having a tropane alkaloid biosynthesis pathway; a seventh objective of the present invention is to provide uses of the hyoscyamine aldehyde reductase, the hyoscyamine aldehyde reductase gene, the recombinant expression vector, and the transgenic cell line or transgenic recombinant bacteria in increasing the content of hyoscyamine in an organism having a tropane alkaloid biosynthesis pathway; and an eighth objective of the present invention is to provide a method for increasing the content of hyoscyamine in a tropane alkaloid synthetic plant.

In order to achieve the above objectives, the present invention provides the following technical solutions:

1. A hyoscyamine aldehyde reductase, being a protein with one of the following amino acid residue sequences:

1) an amino acid residue sequence as shown in SEQ ID NO. 4; and

2) an amino acid sequence having a hyoscyamine aldehyde reductase function obtained by substitution and/or deletion and/or addition of one or more amino acid residues in amino acid residues in SEQ ID NO. 4.

2. A hyoscyamine aldehyde reductase gene, having one of the following nucleotide sequences:

1) a nucleotide sequence as shown in SEQ ID NO. 3;

2) a polynucleotide encoding the nucleotide sequence as shown in SEQ ID NO. 3;

3) a nucleotide sequence having 80% or higher homology with the nucleotide sequence defined in 1) or 2) and encoded to have the hyoscyamine aldehyde reductase function; and

4) a nucleotide sequence hybridizing with the sequence described in 1) or 2).

3. A recombinant expression vector containing the hyoscyamine aldehyde reductase gene.

Preferably, the recombinant expression vector is a recombinant expression vector expressing the hyoscyamine aldehyde reductase and obtained by inserting the hyoscyamine aldehyde reductase gene into a prokaryotic or eukaryotic expression vector.

More preferably, the recombinant expression vector is obtained by ligating the hyoscyamine aldehyde reductase gene having the nucleotide sequence as shown in SEQ ID NO. 3 with a vector pET28a through digestion sites EcoR I and Sac I, or obtained by substituting a GUS gene for transforming pBI121 with the hyoscyamine aldehyde reductase gene having the nucleotide sequence as shown in SEQ ID NO. 3, where transformation of pBI121 is to substitute a 35S promoter of pBI121 with an AbPMT promoter.

4. A transgenic cell line or transgenic recombinant bacteria containing the hyoscyamine aldehyde reductase gene.

Preferably, the cell line is a plant cell line which may be an Atropa belladonna cell line, a plant cell line having a tropane alkaloid biosynthesis pathway, or a plant cell line not having a tropane alkaloid biosynthesis pathway. The recombinant bacteria are BL21 or other bacteria having or not having a tropane alkaloid biosynthesis pathway.

5. A use of the hyoscyamine aldehyde reductase in catalyzing reduction of hyoscyamine aldehyde to produce hyoscyamine in vitro or in vivo.

6. A use of the hyoscyamine aldehyde reductase gene in reconstructing a synthesis pathway of hyoscyamine in a prokaryote or a eukaryote not having a tropane alkaloid biosynthesis pathway.

A use of the recombinant expression vector in reconstructing a synthesis pathway of hyoscyamine in a prokaryote or a eukaryote not having a tropane alkaloid biosynthesis pathway.

7. A use of the hyoscyamine aldehyde reductase in increasing the content of hyoscyamine in an organism having a tropane alkaloid biosynthesis pathway.

A use of the hyoscyamine aldehyde reductase gene in increasing the content of hyoscyamine in an organism having a tropane alkaloid biosynthesis pathway.

A use of the recombinant expression vector in increasing the content of hyoscyamine in an organism having a tropane alkaloid biosynthesis pathway.

A use of the transgenic cell line or transgenic recombinant bacteria in increasing the content of hyoscyamine in an organism having a tropane alkaloid biosynthesis pathway

8. A method for increasing the content of hyoscyamine in a tropane alkaloid synthetic plant, where the hyoscyamine aldehyde reductase gene is over-expressed in a plant having a tropane alkaloid biosynthesis pathway.

The present invention has the beneficial effects that: disclosed in the present invention is a hyoscyamine aldehyde reductase. Research finds that an amino acid sequence of the hyoscyamine aldehyde reductase is as shown in SEQ ID NO. 4 and a nucleotide sequence encoding the hyoscyamine aldehyde reductase is as shown in SEQ ID NO. 3. After prokaryotic expression of the hyoscyamine aldehyde reductase, reduction of hyoscyamine aldehyde can be catalyzed to produce hyoscyamine. After the hyoscyamine aldehyde reductase is used for over-expressing in Atropa belladonna, the content of hyoscyamine in an Atropa belladonna cell line can be increased, which has important significance in increasing the content of tropane alkaloids in Atropa belladonna.

### BRIEF DESCRIPTION OF DRAWINGS

In order to make the objectives, technical solutions and advantages of the present invention clearer, the present invention provides the following drawings for description:

FIG. 1 shows a result of catalyzing reduction of hyoscyamine aldehyde with an HAR to produce hyoscyamine;

FIG. 2 shows expression levels, obtained by fluorescent quantitative PCR detection, of an HAR gene in hairy roots of transgenic Atropa belladonna; and

FIG. 3 shows contents of hyoscyamine in transgenic hairy roots in an HAR over-expression and a control group.

### DETAILED DESCRIPTION OF THE EMBODIMENTS

The embodiments of the present invention are described in detail below. The detailed implementation manners and the specific operation process are provided for the implementation of the embodiments according to the technical solutions of the present invention, but the protection scope of the present invention is not limited to the following embodiments. An experimental method without specific conditions in the embodiments is usually in accordance with conventional conditions, for example, conditions described in Molecular Cloning: A Laboratory Manual of Sambrook, et al. (New York: Cold Spring Harbor Laboratory Press, 1989), or conditions recommended by a manufacturer.

### Embodiment 1. Cloning of Hyoscyamine Aldehyde Reductase (HAR) Gene

(1) Extraction of Total RNA from Atropa belladonna Lateral Roots

A proper amount of Atropa belladonna lateral root tissue was taken, placed and ground up in liquid nitrogen, and added into a 1.5-mL Eppendorf (EP) centrifuge tube containing a lysis solution, and after sufficient mixing of the centrifuge tube, the total RNA was extracted according to an instruction of a TIANGEN kit. The quality of the total RNA was identified with formaldehyde denatured gel electrophoresis, and the concentration of RNA was determined by a spectrophotometer.

(2) Cloning of HAR Gene

With the extracted total RNA as a template, cDNA was synthesized according to an instruction of a TIANGEN FastKing cDNA first strand synthesis kit; and specific primers of the HAR gene were designed. The specific primers were as follows:

HAR-F:
(SEQ ID NO. 1)
5′-atggattcttctggtgtcctct-3′;
and
HAR-R:
(SEQ ID NO. 2)
5′-ttggttgctgctcaaacctag-3′.

The HAR gene was amplified from the total cDNA with PCR and was sequenced, and it was obtained from a sequencing result that a nucleotide sequence of the HAR gene was shown in SEQ ID NO. 3, an initiation codon being ATG and a termination codon being TAA; and a translated protein coding sequence was shown in SEQ ID NO. 4.

### Embodiment 2. Verification of Function of HAR Gene with Prokaryotic Expression

(1) Prokaryotic Expression and Protein Purification of HAR

The HAR gene was amplified with PCR, where a digestion site EcoRI was introduced into a forward primer, and a digestion site SacI was introduced into a reverse primer. A complete sequence in an HAR coding region was ligated to a plasmid pET28a by using the two digestion sites to obtain an HAR prokaryotic expression vector pET28a-HAR. The primers were as follows:

EcoRI-HAR-F:
(SEQ ID NO. 5)
5′-cgcgaattcatggattcttctggtgtcctct-3′;
and
XhoI-HAR-R:
(SEQ ID NO. 6)
5′-cgcctcgagctaggtttgagcagcaaccaa-3′.

The constructed pET28a-HAR plasmid was converted to a prokaryotic expression stain BL21, and a positive clone was screened with PCR to obtain prokaryotic expression engineering bacteria BL21-pET28a-HAR. 100 μL of BL21-pET28a-HAR bacterial liquid was inoculated to a 30 mL of LB liquid medium containing 100 mg/L kanamycin for overnight culture at 37° C. and 200 rpm. Then, a product was respectively inoculated to 400 mL of LB liquid medium according to a ratio of 1 to 50 for activated culture at 37° C. and 200 rpm, and when OD600 was about 0.6, IPTG was added until a final concentration reached 1 mM. Culture was continued for 6 h at 37° C. and 200 rpm. The harvested bacterial liquid was subjected to centrifugation at 8000 rpm, and a supernatant was removed. Then precipitate bacteria were resuspended with a phosphate buffer, subjected to ultrasonication and then purified with a Ni-NTA filler from GE Company to obtain an HAR protein.

(2) Verification on Enzyme Activity of HAR

A hyoscyamine aldehyde reduction reaction system catalyzed by the HAR was as follows: a phosphate buffer (pH 6.4), 0.2 mM NADPH-4Na, 1 mM hyoscyamine aldehyde and 30 μg of HAR protein were incubated for 1 h at 37.5° C., and a reaction product was identified using a high-resolution mass spectrometer.

The high-resolution mass spectrometer selected was Bruker impact II Q-TOF; a chromatographic column selected was a Symmetry C-18 reverse phase silica gel column (3.5 μm, 100×2.1 mm) from Waters Company; a mobile phase A was 0.1% formic acid solution; a mobile phase B was acetonitrile; and gradient elution was employed, and the elution procedure was shown in Table 1:

TABLE 1
Elution Procedure

| Time (min) | Mobile phase A (%) | Mobile phase B (%) |
|---|---|---|
| 0 | 95 | 5 |
| 2 | 75 | 25 |
| 5.5 | 0 | 100 |
| 6.5 | 0 | 100 |
| 6.6 | 95 | 5 |
| 9 | 95 | 5 |

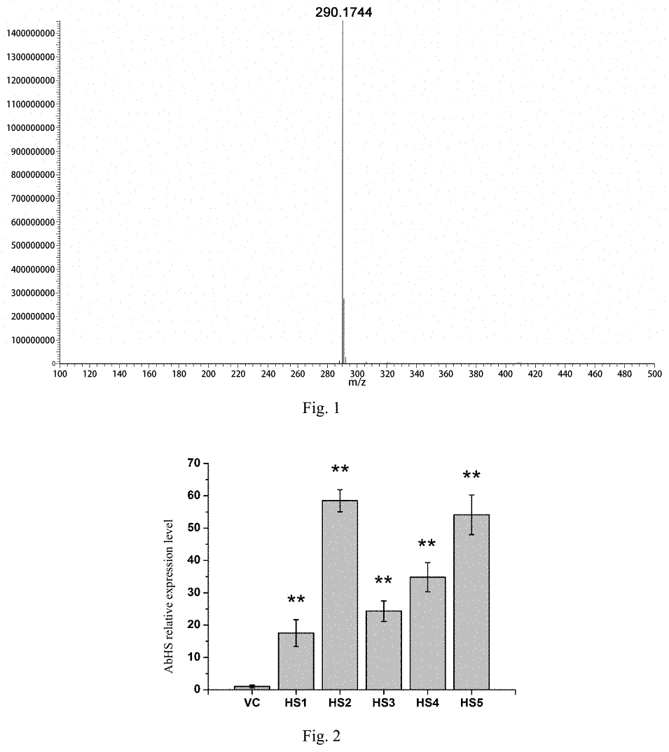

A temperature of the column was set to 40° C.; the flow rate was 0.15 mL/min; the injection volume was 1 μL; and a mass spectrometry detector used an electrospray ion source (ESI) with an ion mode of positive ion mode. A detection result showed that a product of the hyoscyamine aldehyde reduction reaction catalyzed by the HAR was hyoscyamine, a mass-to-charge ratio (m/z) being 290.1744 and a retention time being 4.00 min (FIG. 1).

### Embodiment 3. Increase in Content of Hyoscyamine in Atropa Belladonna with Over-Expression of HAR

(1) Construction of HAR Plant Over-Expression Vector

In order to study the effect of an HAR gene on tropane alkaloids in Atropa belladonna, a pericycle-specific high-expression vector PMT promoter::HAR was constructed, and an original plasmid was pBI121. Firstly, a 35S promoter on the original plasmid pBI121 was substituted with an AbPMT promoter by using digestion sites HindIII and Xba I; and the AbPMT promoter was subjected to PCR amplification with Atropa belladonna cDNA as a template and sequences as shown in SEQ ID NO. 7 and SEQ ID NO. 8 as primers. Then a GUS gene on the original plasmid was substituted with the HAR gene by using digestion sites BamHI and SacI; and the HAR gene was subjected to PCR amplification with Atropa belladonna cDNA as a template and sequences as shown in SEQ ID NO. 9 and SEQ ID NO. 10 as primers to obtain an HAR plant over-expression vector PMT promoter:: HAR. The primer sequences used were as follows:

HindIII-PMT promoter-F:
(SEQ ID NO. 7)
5′-cgcaagcttctgagttcggatctaggtca-3′;
BamHI-PMT promoter-R:
(SEQ ID NO. 8)
5′-cgcggatccttcttcacttttggccttgct-3′;
BamHI-HAR-F:
(SEQ ID NO. 9);
5′-cgcggatccatggattcttctggtgtcctct-3′
and
SacI-HAR-R:
(SEQ ID NO. 10)
5′-cgcgagctcctaggtttgagcagcaaccaa-3′.

(2) Obtaining of Engineering Bacteria of Agrobacterium rhizogenes

The vector PMT promoter::bHAR was transferred into Agrobacterium rhizogenes (such as C58C1) by using a freezing-thawing method and was verified with PCR. A result showed that a plant binary over-expression vector containing the HAR had already been successfully constructed into an Agrobacterium rhizogenes strain.

(3) Obtaining of Transgenic Hairy Roots

A. Preparation of Atropa belladonna Explant

An Atropa belladonna seed was soaked in 75% ethyl alcohol for 1 min, then soaked in 50% NaClO for 20 min and washed with sterile water for 3 to 4 times; the surface of the seed was sipped up with sterile absorbent paper; and the seed was inoculated to a hormone-free ½ MS solid medium for culture under illumination for 16 h/8 h (in the light/dark) at 25° C. to obtain an aseptic seedling of Atropa belladonna. After culture for about 2 weeks under the conditions, a leaf and a hypocotyl explant of the aseptic seedling were sheared for conversion.

B. Co-Culture of Agrobacterium rhizogenes and Explant

The explant was added in an activated resuspension solution (MS+AS 100 μmol/L) of the engineering bacteria of Agrobacterium rhizogenes containing an HAR plant binary over-expression vector, bacterial liquid made full contact with the explant for 5 min and then was transferred onto a co-culture solid medium (MS+AS 100 μmol/L) for dark culture for 2 d at 28° C.

C. Screening of Resistant Hairy Roots

The Atropa belladonna explant subjected to co-culture for 2 d was transferred onto a screening solid medium (MS+Kan 100 mg/L+Cef 500 mg/L) for dark culture at 25° C., subculture was performed once a week, and Kan resistant hairy roots might be obtained after 1 to 2 times of subculture. Well-grown hairy roots were sheared and transferred onto a culture solid medium (MS+Cef 200 mg/L) for culture until completely sterile, and then Kan resistant Atropa belladonna hairy roots were obtained.

D. Genome PCR and Gene Expression Level Assay of Hairy Roots

A forward primer and a reverse primer were respectively designed for detecting a target gene according to a 35S promoter region upstream of an expression cassette and HAR, in which the target gene was located. A result showed that by using the designed PCR-specific primers, a specific DNA fragment could be amplified. However, when a genome DNA of non-converted Atropa belladonna hairy roots was taken as a template, no fragment was amplified.

The obtained transgenic Atropa belladonna hairy roots were subjected to fluorescence quantitative PCR detection; and a result, as shown in FIG. 2, showed that the expression level of an AbHAR gene of the transgenic Atropa belladonna hairy roots was significantly increased.

E. Extraction and Determination of Hyoscyamine in Hairy Roots

The hairy roots were harvested after being subjected to shaking culture for 30 d in an MS liquid medium, and a material was freeze-dried to a constant weight in a freeze dryer. The material was ground into powder, and 0.1 g of plant material which was dried to a constant weight was accurately weighed. 10 mL of alkaloid extraction solution (chloroform:methanol:aqueous ammonia=15:5:1) was added, ultrasonic extraction was performed for 30 min, and a product was allowed to stand for 1 h at a room temperature. The extraction solution was filtered to remove plant residues, and a filtrate was dried at 40° C. The dry matter in the last step was dissolved with 5 mL of chloroform and 2 mL of 0.5 M sulfuric acid and was fully emulsified to enable alkaloids to be transferred into an aqueous phase, and the chloroform was discarded. The aqueous phase was placed on ice, and a pH value of the aqueous phase was adjusted with aqueous ammonia (28%) to 10.0. 2 mL of chloroform was added for extracting the alkaloids, the extraction was repeated for two times, all the chloroform was combined, dried with anhydrous sodium sulfate to remove water, and filtered, and a filtrate was dried at 40° C. The alkaloids were dissolved with 1 mL of liquid chromatography grade methanol; the solution was filtered with a filter membrane of 0.22 μm; the content of the alkaloids was determined with HPLC; and a result was an average of three repeated experiments, where an error bar indicated a standard deviation. Statistical analysis adopted a t-test.

Configuration of an HPLC instrument: Shimadzu “Prominence” LC-20AD binary pump system, equipped with a DUG-20A online degasser, a CTO-20A column oven, an SPD-M20A full-wavelength diode array detector, an SIL-20A automatic sampler, a chromatographic column of Shimadzu INERTSUSTAIN C18 chromatographic column (5 μm, 4.6×250 mm) and a Shimadzu guard column (5 μm, 4.0×10 mm).

Chromatographic conditions for alkaloid analysis: the mobile phase adopted 11% acetonitrile and 89% water (20 mM ammonium acetate and 0.1% formic acid, pH 4.0), a temperature of the column oven was set to 40° C., a total flow rate was 1 mL/min, and a detection wavelength was 226 nm.

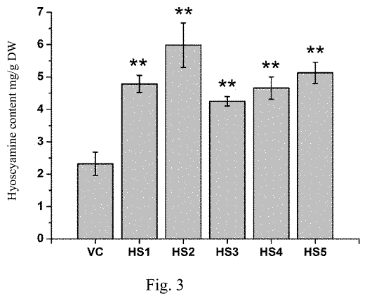

A result, as shown in FIG. 3, showed that, in the present invention, the content of hyoscyamine in transgenic hairy roots of a AbHAR over-expression had been significantly increased. When the content of hyoscyamine in ordinary hairy roots is 2.32 mg/g DW, an average content of hyoscyamine in Atropa belladonna hairy roots of an AbHAR over-expression in the same period reached 4.24-5.98 mg/g DW, and the content was 1.83-2.58 times that in non-transgenic Atropa belladonna hairy roots.

The above embodiments are listed preferred embodiments only for full description of the present invention, and the protection scope of the present invention is not limited thereto. Equivalent substitutions or transformations made by those skilled in the art according to the present invention are within the protection scope of the present invention. The protection scope of the present invention is subject to the protection scope defined by the claims.

1. A hyoscyamine aldehyde reductase, being a protein with one of the following amino acid residue sequences:
1) an amino acid residue sequence as shown in SEQ ID NO. 4; and
2) an amino acid sequence having a hyoscyamine aldehyde reductase function obtained by substitution and/or deletion and/or addition of one or more amino acid residues in amino acid residues in SEQ ID NO. 4.

2. A hyoscyamine aldehyde reductase gene, having one of the following nucleotide sequences:
1) a nucleotide sequence as shown in SEQ ID NO. 3;
2) a polynucleotide encoding the nucleotide sequence as shown in SEQ ID NO. 3;
3) a nucleotide sequence having 80% or higher homology with the nucleotide sequence defined in 1) or 2) and encoded to have the hyoscyamine aldehyde reductase function according to claim 1; and
4) a nucleotide sequence hybridizing with the sequence described in 1) or 2).

3. A recombinant expression vector containing the hyoscyamine aldehyde reductase gene according to claim 2.

4. The recombinant expression vector according to claim 3, wherein the recombinant expression vector is a recombinant expression vector for expressing the hyoscyamine aldehyde reductase and obtained by inserting the hyoscyamine aldehyde reductase gene into a prokaryotic or a eukaryotic expression vector.

5. A transgenic cell line or transgenic recombinant bacteria containing the hyoscyamine aldehyde reductase gene according to claim 2.

6. The transgenic cell line according to claim 5, wherein the cell line is a plant cell line.

7. A use of the hyoscyamine aldehyde reductase according to claim 1 in catalyzing reduction of hyoscyamine aldehyde to produce hyoscyamine in vitro or in vivo.

8. A use of the hyoscyamine aldehyde reductase gene according to claim 2 in reconstructing a synthesis pathway of hyoscyamine in a prokaryote not having a tropane alkaloid biosynthesis pathway.

9. A use of the hyoscyamine aldehyde reductase gene according to claim 2 in reconstructing a synthesis pathway of hyoscyamine in a eukaryote not having a tropane alkaloid biosynthesis pathway.

10. A use of the recombinant expression vector according to claim 3 in reconstructing a synthesis pathway of hyoscyamine in a prokaryote or a eukaryote not having a tropane alkaloid biosynthesis pathway.

11. A use of the recombinant expression vector according to claim 4 in reconstructing a synthesis pathway of hyoscyamine in a prokaryote or a eukaryote not having a tropane alkaloid biosynthesis pathway.

12. A use of a transgenic cell line or transgenic recombinant bacteria containing the hyoscyamine aldehyde reductase according to claim 1 in increasing the content of hyoscyamine in an organism having a tropane alkaloid biosynthesis pathway.

13. A use of a transgenic cell line or transgenic recombinant bacteria containing the hyoscyamine aldehyde reductase gene according to claim 2 in increasing the content of hyoscyamine in an organism having a tropane alkaloid biosynthesis pathway.

14. A use of a transgenic cell line or transgenic recombinant bacteria containing the recombinant expression vector according to claim 3 in increasing the content of hyoscyamine in an organism having a tropane alkaloid biosynthesis pathway.

15. A use of a transgenic cell line or transgenic recombinant bacteria containing the recombinant expression vector according to claim 4 in increasing the content of hyoscyamine in an organism having a tropane alkaloid biosynthesis pathway.

16. A use of the transgenic cell line or transgenic recombinant bacteria according to claim 5 in increasing the content of hyoscyamine in an organism having a tropane alkaloid biosynthesis pathway.

17. A method for increasing the content of hyoscyamine in a tropane alkaloid synthetic plant, wherein the hyoscyamine aldehyde reductase gene according to claim 2 is over-expressed in a plant having a tropane alkaloid biosynthesis pathway.
